(19) United States
(12) Patent Application Publication
(10) Pub. No.: US 2026/0083511 A1
(43) Pub. Date: Mar. 26, 2026

(54) Foot Ankle Reference Body, as well as Ground Plate and Arms for the same

(71) Applicant: Stryker European Operations Limited, Carrigtwohill (IE)

(72) Inventors: Jesse Moore, Germantown, TN (US); Johnny McGee, Halls, TN (US)

(21) Appl. No.: 19/108,661

(22) PCT Filed: Sep. 5, 2022
(86) PCT No.: PCT/EP2022/074648

§ 371 Date: Mar. 4, 2025

(51) Int. Cl. A61B 34/20; A61B 90/00; G16H 40/63

(52) U.S. Cl. CPC A61B 34/20; A61B 90/39; G16H 40/63; A61B 2034/2065; A61B 2034/2072; A61B 2090/376; A61B 2090/3916; A61B 2090/3937; A61B 2090/3966

(57) ABSTRACT

Reference body and corresponding method for providing a reproducible reference to predetermined reference axes of a virtual coordinate system for fluoroscopic/x-ray imaging comprising a radio dense central marker located at a center of the virtual coordinate system, a plurality of radio dense main satellite markers each located on one of the main axes of the virtual coordinate system, so that a line between the center marker and one of the main satellite markers represent a main axis of the virtual coordinate system for reducing imaging effort and thus duration of the surgery, radiation impact on the patient, while maintaining exactness of the surgery.

### FIELD OF THE INVENTION

The present invention relates to a reference body for providing a reproducible reference to predetermined reference axes of a virtual coordinate system for fluoroscopic/x-ray imaging, as well as a reference body ground plate and a reference body arm of a reference body for providing a reproducible reference to predetermined reference axes of a virtual coordinate system for fluoroscopic/x-ray imaging.

### TECHNICAL BACKGROUND OF THE INVENTION

Surgical procedures have improved over the recent years. Significant improvements have been achieved by supporting systems for supporting the clinical personal in particular a surgeon during surgeries. In particular bone fractures benefit from supporting systems for surgeons, which provide the surgeon with equipment, which allows the surgeon to improve exactness of repositioning of bone parts and positioning of implants, like screws, nails and bone plates, as well as tools and targeting and guiding devices.

As traumatized bones, i.e., fractures, have only a limited visual access, monitoring is usually based on radiating principles, like X-ray imaging or computer tomography CT images, or magnet resonance tomography MRT images. All these principles and methods involve at least one of the drawbacks of being radiation intensive, requiring large devices and requiring a considerable amount of time. Each monitoring step during a surgery prolongs the surgery duration and thus the duration of narcotic impact and increases costs and radiation impact.

Therefore, there is a need for surgical reference bodies, which reduce imaging effort and thus duration of the surgery, reduce radiation impact on the patient, but at the same time maintain or increase the level of exactness of the surgery.

### SUMMARY OF THE INVENTION

The present invention provides a reference body, a ground plate for said reference body and an arm for said reference body allowing an improved localization during surgery according to the subject matter of the independent claims. Further embodiments are incorporated in the dependent claims.

According to an embodiment there is provided a reference body for providing a reproducible reference to predetermined reference axes of a virtual coordinate system for fluoroscopic/x-ray imaging, the reference body comprises: a radio dense central marker located at a center of the virtual coordinate system, a plurality of radio dense main satellite markers each located on one of the main axes of the virtual coordinate system, so that a line between the center marker and one of the main satellite markers represent a main axis of the virtual coordinate system.

This allows a defined determination of a relative position of an anatomy with respect to the reference body. The position of radio dense satellite marker on reproducible axes corresponding to a virtual coordinate system meeting at a center point where also the radio dense center marker is located allows a reproducible reference to said coordinate system. In general, the coordinate system may be any coordinate system, like a Cartesian coordinate system. The axes do not have to be orthogonal to each other but may have any angle as far as it spans a tree dimensional space. The representation of the axes by the line between the center marker and one of the satellite markers allows an immediate reference to said coordinate system and simplifies the calculation of the relative position of an anatomy with respect to navigating coordinates. The main satellite markers are markers along the main axes of the virtual coordinate system, e.g., axes x, y and z. Sub-satellite markers or auxiliary satellite markers are markers along sub axes or auxiliary axes of the virtual coordinate system, like axes corresponding to bisecting lines between two or three of the main axes.

The reference body may have a carrier structure which carries the radio dense satellite markers and the center marker. The carrier structure may be of one piece or separable along one or more separation interfaces. The latter allows assembling around an anatomy if no access is given to the entire reference body. The interfaces may be designed to allow only a predefined assembly in order to maintain the relative position of the center marker and the satellite markers with respect to each other.

According to an embodiment the virtual coordinate system is a Cartesian coordinate system with orthogonal x, y and z axes, wherein each of the main satellite marker are provided on the positive branch of the main axes of x, y and z axes.

Thus, a common coordinate system may be applied as a virtual coordinate system and the calculation with respect to the positions and directions is easy. By providing a main satellite marker on each of the positive branches allows spreading a spatial section of one eighth of a sphere. Each coordinate can be provided as a positive amount or value, which simplified handling of the position data and makes relative positions transparent.

According to an embodiment the reference body further comprises a plurality of sub satellite markers each provided on an equally divided angles between two positive branches out of the x, y and z axes, wherein in particular the equally divided angle is 45 deg or 30 deg.

Thus, not only satellite markers on the main axes can be provided, but also satellite markers on auxiliary axes or sub axes. The auxiliary axes may be provided in equally divided angles, so that each of the equally divided angles have the same size and together result in the angle of two main axes. This is for example the case when providing an auxiliary axis as a bisecting line between two main axes resulting in a 45 deg angle. As an alternative the auxiliary axes may be equally spaced by 30 deg or 15 deg between two main axes. It should be noted that also a sub marker can be provided along an auxiliary axis which is equally distant intersected between all three main axes.

According to an embodiment at least a part, in particular all satellite markers are equal distant from the center marker.

Thus, the satellite markers can be provided on a shell of a sphere, which provides a good spatial overview and recognition in an x-ray or fluoroscopic image.

According to an embodiment the center marker has a radio dense shape of a spatial cross with cross lines along the main axes of the virtual coordinate system.

Thus, a cross shape or a spatial cross shape may have a preference direction where the cross arms extend toward predefined satellite markers, in particular toward main satellite markers on main axes of the virtual coordinate system, e.g. axes x, y and z. As an alternative, the center marker may also have a radio dense ball shape if it is desired to have not preference direction.

According to an embodiment at least one of the satellite markers has a radio dense shape of a ring with a ring opening aligned to center marker and/or a radio dense shape of cross of an extension traverse to the direction toward the center marker.

Thus, it is possible to use the satellite marker, be it a main satellite marker or a sub or auxiliary satellite marker as a targeting tool, where the center marker can be brought into correspondence with the central opening of the ring, if the viewing direction is exact along the corresponding axis along which the satellite marker is positioned.

According to an embodiment at least a part of the satellite marker has locally allocated optical markers, wherein in particular each of the optical markers has a unique optical pattern allowing identification as well as determination of a position of the respective satellite markers.

Thus, it is possible to match a fluoroscopic or x-ray image with an optical image. Once the correspondence is established, the navigation can be carried out by optical imaging rather than x-ray imaging, so that the radiation impact can be reduced. Optical image means imaging a wavelength range which is perceptible by a human being. The unique pattern allows determination of the orientation and position based on a single optical image.

According to an embodiment the reference body further comprises a reference body ground plate and a plurality of reference body arms, wherein the center marker is fixed to the ground plate at a predefined center position corresponding to a center point of the virtual coordinate system, wherein each of the plurality of arms has a linear guiding portion at the one end of one of the plurality of arms, wherein one of the satellite markers is provided in a predefined orthogonal distance from the linear extension of the linear guiding portion at the other end of the respective one of the plurality of arms, wherein the ground plate comprises a plurality of receptacles with a linear extension for receiving a respective linear guiding portion of one of the arms, wherein each of the linear extensions of the receptacles is offset by a predefined orthogonal distance from the center position of the center marker so that a satellite marker positioned at the respective predefined orthogonal distance from the linear extension of the linear guiding portion is displaceable along a linear trajectory running through the center position of the center marker.

Thus, it is possible to adjust the satellite marker with respect to their distance from the central marker, however without leaving the respective axis of the virtual coordinate system. The satellite markers are mounted on arms, wherein the arms have linear guiding, which trajectory may be parallel offset, i.e. in an orthogonal distance offset along the respective axis of the virtual coordinate system. The orthogonal distance is the smallest distance of a point from a line, which is the distance along a line which stands orthogonal to the distant line. A movement of the arm along the linear guidance leads to a shift of the marker along the respective axis, however without leaving this axis. The arm between the linear guidance portion and the position of the marker may have any form, in particular can be bent or curved. This allows an alignment of the satellite marker with the center marker along the respective axis of the virtual coordinate system, even if the anatomy extends through the respective axis. As the center marker is fixed to the ground plate, shifting the arms only changes the distance of the satellite maker from the center marker, but not the orientation of the line between the center marker and the respective satellite marker.

According to an embodiment each of the receptacles has an unmistakable cross sectional shape representative for an orthogonal distance of the center marker from the linear extension of said receptacle for unmistakable receiving a linear guiding portion of one of the reference body arms with a corresponding cross sectional shape representative for an orthogonal distance of the satellite marker on said arm from the linear extension of the linear guiding portion.

Thus, it can be ensured that only those arms are connected to a corresponding receptacle of the ground plate. Thus, it can be ensured, that only an arm is coupled to the guiding portion of the ground plate in a key/keyhole manner, where the orthogonal distance of a marker to a linear trajectory line of the guiding portion of the arm corresponds to the orthogonal distance of the center marker on the ground plate to the trajectory line of the receptacle in the ground plate. This results in only a change of distance of the satellite marker from the center marker, but not in a change of the orientation of the line between the satellite marker and the center marker.

According to an embodiment the receptacles are through bores along axes for distance adjustment of the respective satellite marker on the respective reference body arm.

Thus, the arms can be adjusted according to a distance of the satellite marker from the center marker. It should be noted that fixing items can be provided in order fix the linear guiding portion in any desired position corresponding to a distance of the satellite marker from the center marker. Adjusting the distance of the arms allows for ingress, restraint, and egress of the patient for fluoroscopic imaging.

According to an embodiment the receptacle and the reference body arm has a corresponding color code representative for the orthogonal distance of the center portion from a linear extension of the receptacle and the satellite marker from the linear extension of the linear guiding portion of the reference body arm.

Thus, it is easy perceivable by a surgeon which arm can be used for which receptacle. It should be noted that arms can be exchanged or put into different receptacle, as long as the orthogonal distance of the trajectory line of the receptacle to the center marker corresponds to the orthogonal distance of the satellite marker to the trajectory line of the guiding portion of the arm. It should be noted that the orthogonal distance can be the same for all receptacles on the ground plate, so that a matching arm can be used for each of the receptacles and for each of the receptacles has the effect of changing only the distance of the satellite marker to the center marker, but not the orientation of their connecting line.

According to an embodiment the reference body arm comprises an additional radio marker arrangement with a line marker along the axis of a linear extension of the linear guiding portion and a ring marker concentric to the axis of the linear extension of a linear guiding portion, and distant from the line marker along the axis of the linear extension of a linear guiding portion.

Thus, it is possible finding exact the viewing direction onto the reference body, which corresponds to the respective axis of the virtual coordinate system. If additionally providing the satellite marker in a ring shape, as describes above, both targeting possibilities can be compared in order to verify that the targeting is reliable. In this case the ring shape satellite marker has the center marker in the center of the ring and at the same time the ring at the arm matches with the front view of the line marker, which is e.g., a dot.

According to an embodiment there is provided a reference body ground plate, the ground plate comprises a radio dense center marker located at center position representing a center of a virtual coordinate system, a plurality of receptacles with a linear extension for receiving a linear guiding portion of an reference body arm to be connected to the ground plate, wherein each of the linear extensions of the receptacles is offset parallel in a predefined orthogonal distance from one of the main axes of the virtual coordinate system, so that a satellite marker positioned at a respective predefined orthogonal distance from the extension of the linear guiding portion of a reference body arm to be connected to the ground plate is displaceable along the respective main axis of the virtual coordinate system.

Thus, a ground plate is provided for a reference body describe above.

According to an embodiment each of the receptacles has an unmistakable cross sectional shape representative for an orthogonal distance of the center marker from the linear extension of said receptacle for unmistakable receiving a linear guiding portion of a reference body arm with a corresponding cross sectional shape representative for an orthogonal distance of a satellite marker on said reference body arm from a linear extension of the linear guiding portion of said reference body arm to be connected to the reference body ground plate.

Thus, a surgeon can physically use only those arms for a particular receptacle, where the orthogonal distance matches as describes above. It should be noted that arms with a particular cross section of the guiding portion can be allocated to different receptacle having the same cross sectional receptacle, as long as the orthogonal distance of the trajectory line of the receptacle to the center marker corresponds to the orthogonal distance of the satellite marker to the trajectory line of the guiding portion of the arm. It should be noted that the orthogonal distance and thus the cross section can be the same for all receptacles on the ground plate, so that a matching arm can be used for each of the receptacles and for each of the receptacles has the effect of changing only the distance of the satellite marker to the center marker, but not the orientation of their connecting line.

According to an embodiment the receptacles are through bores along axes, e.g., x, y, z, for distance adjustment of a satellite marker on a reference body arm to be connected to the reference body ground plate.

Thus, arms can be adjusted according to a distance of the satellite marker from the center marker. It should be noted that fixing items can be provided in order fix the linear guiding portion in any desired position corresponding to a distance of a satellite marker from the center marker.

According to an embodiment the receptacle and a reference body arm to be connected have a corresponding color code representative for the orthogonal distance of the center portion from a linear extension of the receptacle and a satellite marker from a linear extension of a linear guiding portion of a reference body arm to be connected.

Thus, it is easy perceivable by a surgeon which arm can be used for which receptacle in the ground plate, as describe above with respect to the entire reference body.

According to an embodiment there is provided a reference body arm for positioning a satellite marker with respect to a ground plate to which the reference body arm is to be connected, the reference body arm comprises a linear guiding portion to be received by a receptacle of a ground plate, a radio dense satellite marker positioned at a predefined orthogonal distance from a linear extension of the linear guiding portion of the reference body arm.

Thus, an arm can be provided for being used in connection to the above-described ground plate in order to work as a reference body as described above.

According to an embodiment the linear guiding portion has an unmistakable cross sectional shape representative for the orthogonal distance of the satellite marker from the linear extension of the linear guiding portion for unmistakable to be received by a linearly extending receptacle of a reference body ground plate with a corresponding cross sectional shape representative for a corresponding orthogonal distance of a linear extension of the linearly extending receptacle from a center portion of the reference body ground plate to which the reference body arm is to be connected.

Thus, a surgeon can physically match only those arms with a respective receptacle in the ground plate which has a corresponding orthogonal distance, as described above with respect to the entire reference body.

According to an embodiment the reference body arm and a receptacle to which the reference body arm is to be connected have a corresponding color code representative for the orthogonal distance of the satellite marker from the linear extension of the linear guiding portion of the reference body arm and a center portion of a reference body ground plate to which the reference body arm is to be connected from a linear extension of a receptacle of a reference body ground plate to which the reference body arm is to be connected.

Thus, it is easy perceivable by a surgeon which arm can be used for which receptacle in the ground plate, as describe above with respect to the entire reference body.

According to an embodiment the reference body further comprises an additional radio marker arrangement with a line marker along the axis of a linear extension of the linear guiding portion and a ring marker concentric to and distant along the axis of the linear extension of a linear guiding portion from the line marker.

Thus, it is possible finding exact the viewing direction onto the reference body, which corresponds to the respective axis of the virtual coordinate system, as describe above with respect to the entire reference body.

It should be noted that the above-described embodiments may also be combined and in a combined form provide a synergetic technical effect and synergetic benefits which go beyond the sum of the single technical effects and benefits.

### BRIEF DESCRIPTION OF THE FIGURES

The invention will be described by way of the following drawings, which illustrate in

FIG. 1: illustrates an outer view onto a reference body having a number of radio dense markers according to an exemplary embodiment of the invention;

FIG. 2: illustrates main axes and auxiliary or sub-axes of a coordinate system with respect to satellite radio dense markers of a reference body according to an exemplary embodiment of the invention;

FIG. 3: illustrates an outer view onto a reference body having a number of radio dense markers with an optical pattern according to an exemplary embodiment of the invention;

FIG. 4: illustrates an outer view onto a reference body having a number of radio dense markers with another kind of unique optical pattern according to an exemplary embodiment of the invention;

FIG. 5: illustrates an inner view onto a reference body having a number of radio dense markers and a center marker according to an exemplary embodiment of the invention;

FIG. 6: illustrates main axes and auxiliary or sub-axes of a coordinate system meeting at a center point with respect to satellite radio dense markers of a reference body according to an exemplary embodiment of the invention;

FIG. 7: Illustrates a reference body according to FIG. 5 applied to an anatomy;

FIG. 8: illustrates an outer view onto a reference body with a ground plate and a number of arms having a center marker and a number of radio dense markers according to an exemplary embodiment of the invention;

FIG. 9: illustrates a reference body according to FIG. 8 applied to an anatomy;

FIG. 10: illustrates different guiding portions of arms with different cross sections according to an exemplary embodiment of the invention.

FIG. 11: illustrates a line marker and a ring marker in a viewing direction aligned to the arm orientation according to an exemplary embodiment of the invention.

FIG. 12: illustrates the reference body relative to the anatomy and an implanted implant.

FIG. 13: illustrates schematically an x-ray view with overlapping images of an anatomy with implanted implant and the reference body with radio dense or radiopaque markers and features in a simulated monitor screen projection.

It should be noted that same or similar reference numerals illustrate same or similar components. Along these Figures exemplary embodiments of the invention will be describes as follows.

### DETAILED DESCRIPTION OF EXEMPLARY EMBODIMENTS

The present invention provides a reference body, a ground plate for a reference body and an arm of a reference body, which provide a number of radio markers which allow determination and reference to predetermined axes. A radio dense center marker and a number of outer radio dense satellite markers are arranged in space so that each outer satellite marker is in line with the center marker along a predetermined axis, e.g., x, y, z axes of a Cartesian coordinate system and optionally predetermined angles there between (45 deg, 30/60 deg). This allows referencing in a fluoroscopic image. Fluoroscopic images may be taken at multiple discrete angles, including perspective, isometric, and axonometric views, which will aid the 3D model approximation.

The idea behind is to help a computer system to compile few or several (not thousands like a CT scan) discrete 2D images at different angles for an estimated 3d model. Having a central coordinate center and axes which is necessary for relative calculations. This is a simple structure and method of adding markers into x-rays for digital size and angle calibrations.

This is particularly useful for identifying the actual angle of the x-ray when it is not clear to a human eye. For example, if an x-ray is taken at 12, 9, and 58 degrees in the corresponding x, y, and z axes, it can be detected by the reference point and coordinate system markers to calculate for 3D model orientation and a best fit approximation given a fluoroscopic image input taken at any angle.

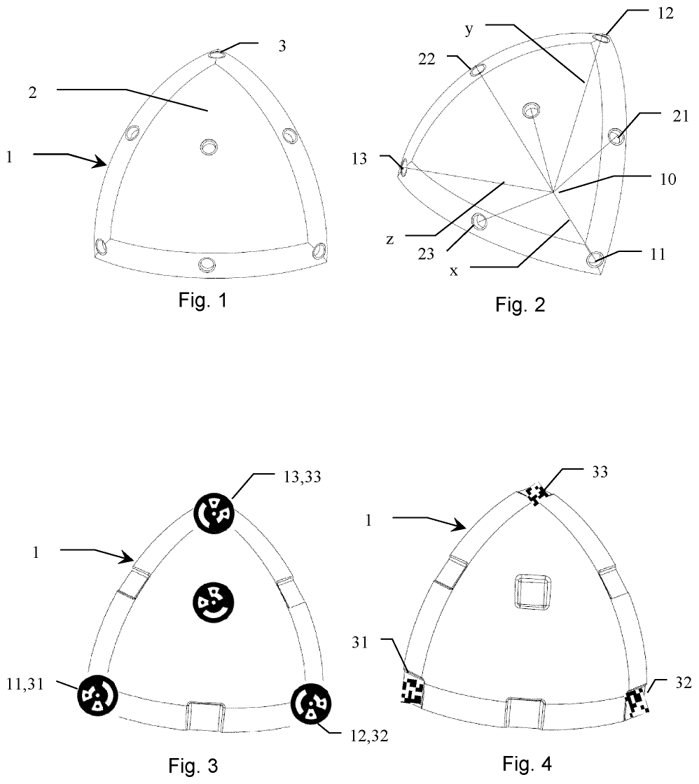

For this purpose, a reference body is provided, which is illustrated in one or more of FIGS. 1 to 7. The idea is to provide a portable e.g., semi-sphere device used to co-ordinate fluoroscopically aligned markers in the orientation of each view taken from any direction with the same semi-sphere. Putting the semi-sphere in the field of view of the anatomy at various angles during x-ray allows for one central radiographic reference point. For example, in the sagittal plane, views can be angularly coordinated with a 0-, 45-, and 90-degree view as well as with 0-, 45-, and 90-degree in the frontal plane. One coordinate system allows for precise reference and interpolation of the 2D image taken along a 3D axis. This is particularly useful when optical systems align 2D images into 3D models, wherein discrete 2D images may be used to orient position and scale with an existing 3D model in software. Further, the discrete 2D images may be utilized to compute an approximation of a 3D model for a unique patient. The semi-sphere may also indicate optically, using planar fiducial markers or reflective spheres, where the x-ray machine should align before a 2D image is made. This can also interact with AR and MR algorithms to coordinate location in 3D space. Fluoroscopic markers may include a ball and circle centering site, or any pattern of spheres, pins, and circles to coordinate visual cues along an axis for various 2D fluoroscopic images. Corresponding embodiments will be described in the following.

A further idea, which can be considered as a further development of what is described with respect to FIGS. 1 to 7 is a reference body operating and serving as a portable foot wrapping system with built in fluoroscopic alignment markers to indicate the orientation of each 2D x-ray view taken in 3D. Alignment markers are removable and adjustable in an offset position along a predetermined axis to align fluoroscopic view finders. Optical coordination is also enabled with added planar fiducial markers for reference. This same idea could be built into a reference body in the shape of an “x-ray shoe”, as illustrated e.g. in FIG. 7, 8 or 9.

FIG. 1 illustrates an outer view onto a reference body 1 having a number of radio dense markers or receptacles 3 for a radio dense marker according to an exemplary embodiment of the invention. FIG. 2 illustrates main axes x, y, z and auxiliary or sub-axes of a coordinate system with respect to satellite radio dense markers 11, 12, 13 of the reference body 1 according to an exemplary embodiment of the invention. The reference body 1 may have a carrier structure 2 for carrying the center marker 10 and the satellite markers 11, 12, 13, 21, 22, 23. The satellite marker may be main satellite markers 11, 12, 13, which are positioned along main axes x, y, z od a virtual coordinate system, which is here a Cartesian coordinate system. It should be noted that also every other coordinate system may be applied. In addition to the main satellite markers 11, 12, 13 also auxiliary or sub markers 21, 22, 23 can be provided which are not aligned to the main axes x, y, z of the virtual coordinate system, but to auxiliary axes or sub axes of the virtual coordinate system. In the embodiment illustrated in FIG. 2, the auxiliary axes for the auxiliary markers 21, 22, 23 are bisecting axes of each two main axes x, y, z of the virtual coordinate system. All axes intersect in the position of the center marker 10, which is a center point CP of the virtual coordinate system. FIG. 2 can be understood as an illustration of a frame or carrier structure 2 with receptacles 3 for satellite markers, 11, 12, 13, 21, 22, 23, or as reference body with already received satellite markers 11, 12, 13, 21, 22, 23. The reference body then may be used as a reference providing a coordinate system in a fluoroscopic or x-ray image, as the radio dense satellite markers are always in predefined axes, be it main axes or auxiliary axis of a coordinate system. In case the distance of all satellite markers from the center marker 10 is the same, identification is even easier. The reference body 1 may be used as a compact easy to handle device, which can be supplemented to an x-ray imaging environment for providing a coordinate system reference. It is understood that the reference body 1 may be utilized in many scenarios, such as prior to surgery as surgical planning tool, during surgery as a coordinating and live plan modification tool, and also after surgery as a diagnostic and measurement aid. The reference body 1 may be placed in view of any imaging modality to provide a universal scale and orientation between phases of patient progression. It may be used with any bone or set of bones in fluoroscopic imaging.

In some cases, it may be helpful bringing an optical image in the visible wavelength range into conformity with an x-ray image, e.g., for navigation at least partially upon optical imaging instead of x-ray imaging. FIG. 3 illustrates an outer view onto a reference body 1 having a number of radio dense markers 11, 12, 13 each with an optical pattern 31, 32, 33 according to an exemplary embodiment of the invention. The optical pattern may be located in direct correspondence to the respective radio dense marker 10, 11, 12, 13. Here illustrated are the satellite marker, in particular the main satellite marker 11, 12, 13. However, also the auxiliary satellite marker 21, 22, 23 and the center marker can be provided with an optical marker in form of an optical pattern. The pattern 31, 32, 33 illustrated in FIG. 3 allows an easy determination of the orientation of the marker 11, 12, 13 and thus the entire reference body 1. In some cases, it may be helpful to identify a particular satellite marker. For this purpose, the optical pattern of the optical marker 31, 32, 33 may be a unique optical pattern. FIG. 4 illustrates an outer view onto a reference body 1 having a number of radio dense markers 11, 12, 13 having allocated optical marker 31, 32, 33 with another kind of unique optical pattern according to an exemplary embodiment of the invention. The optical marker can be provided with e.g., a QR-code-like pattern, which allows not only identification of a particular optical marker 31, 32, 33, but also the determination of its spatial position and orientation.

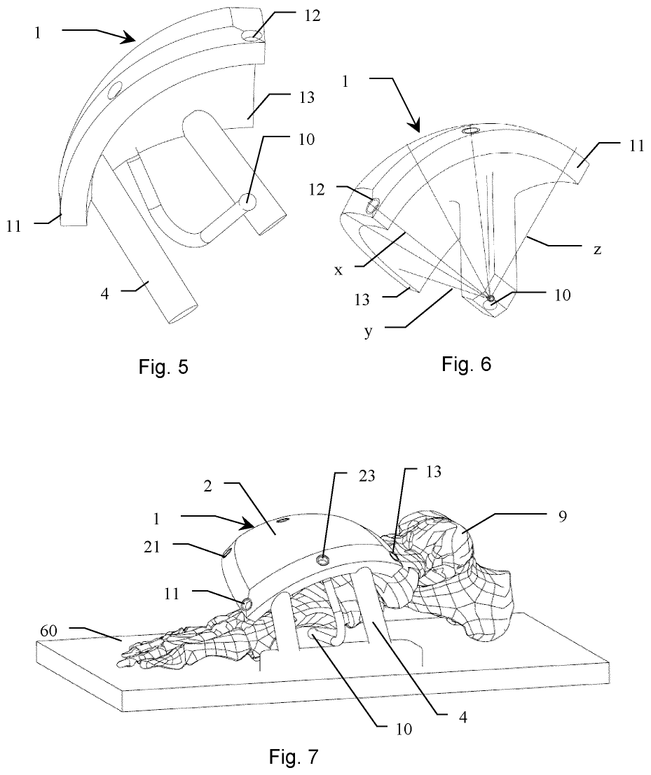

FIG. 5 illustrates an inner view onto a reference body 1 having a number of radio dense satellite markers 11, 12, 13 and a center marker 10 according to an exemplary embodiment of the invention. The satellite markers 11, 12, 13 in this embodiment are located on a sphere, to be exact, on one eighth of a sphere. The center marker 10 is provided on an extension, which positions the center marker 10 in a center point of a here Cartesian coordinate system, as describe in connection with FIGS. 1 and 2. The reference body 1 is further provided with a support structure 4 which allows a standing position of the reference body 1. The support structure 4 here is fixedly connected to the carrier structure 2, however, without departing from the purpose and effect of the invention, the support structure 4 can also be connected to the carrier structure 2 in a hinged or flexible way, in order to adjust the orientation of the virtual coordinate system and respectively the center marker 10 and the satellite marker 11, 12, 13, 21, 22, 23 relative to an underground onto which the reference body 1 is to be positioned.

There may be a desire to combine the support structure 4 with a post for the center marker 10, in order to make the reference body 1 more robust and compact. FIG. 6 illustrates such a reference body 1 with main axes x, y, z and auxiliary or sub-axes of a coordinate system meeting at a center point 10 with respect to satellite radio dense markers 11, 12, 13 of a reference body 1 according to an exemplary embodiment of the invention. Compared with the embodiment illustrated in FIG. 5, the support structure 4 in FIG. 6 is also used for receiving the center marker 10. It should be noted that the center marker 10 may be designed as an exchangeable center marker 10, in order to provide upon need a ball shape center marker or a (spatial) cross shape center marker 10. In case of a cross shape center marker, the receptacle may be designed to receive the center marker 10 in predefined orientations matching e.g., the main axes x, y, z.

FIG. 7 illustrates the reference body 1 according to FIG. 5, which is applied to an anatomy 9. The support structure 4 of the reference body 1 serves for mounting the carrier structure 2 of the reference body 1 to a ground plate 60 thereof. Although not illustrated in detail, the reference body carrier structure 2 may be mounted in a variable position and orientation with respect to the ground plate 60. The reference body 1 with the applied exemplary anatomy 9 may support a surgeon upon orientation during surgery, as the reference body 1 provides a reference geometry and in particular a reference coordinate system with respect to the anatomy 9.

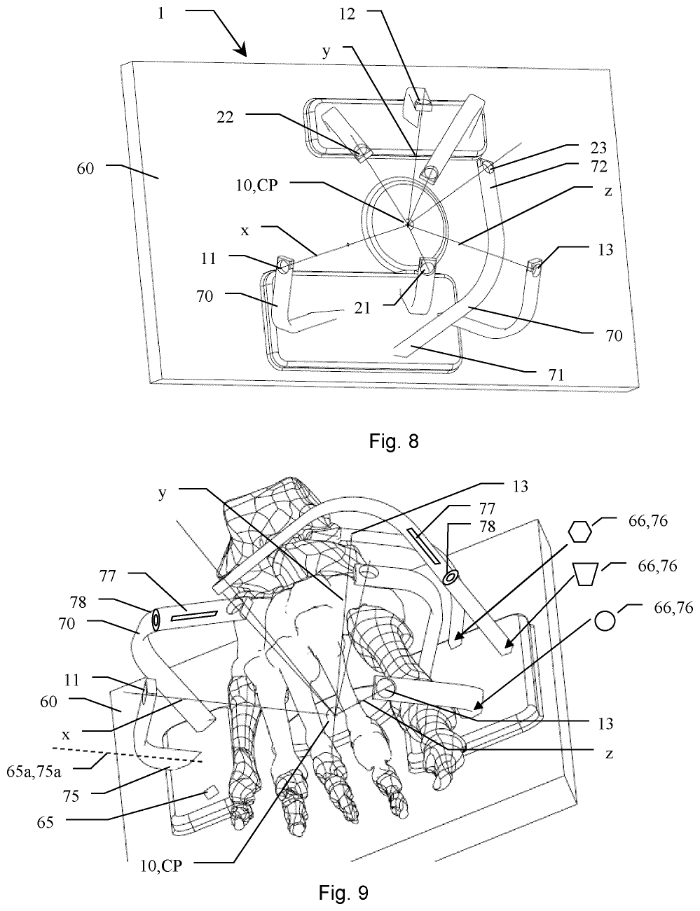

FIGS. 1 to 7 illustrate a reference body 1, where the satellite markers 11, 12, 13, 21, 22, 23 are fixedly mounted with respect to each other. With complex anatomies 9, there may be a desire providing more flexibility with respect to the space and also during positioning of the anatomy. FIG. 8 illustrates an outer view onto a reference body 1 with a ground plate 60 and a number of arms 70 having a center marker 10 at a center point CP of a virtual coordinate system with axes x, y, z and a number of radio dense satellite markers 11, 12, 13, 21, 22, 23 according to an exemplary embodiment of the invention, which allows more flexibility. The reference body arms 70 each have a first end 71 which is connected or connectable to the ground plate 60 and a second end 72 to which a satellite marker 11, 12, 13, 21, 22, 23 is mounted or mountable. Any releasable mounting between the first end 71 of arm 70 and the ground plate 60 as well as the second end 72 of the arm 70 and the satellite marker 11, 12, 13, 21, 22, 23 may be designed as an interface allowing only predefined orientations and positions. The main satellite markers 11, 12, 13 are positioned by the arms so that they are positioned along one of the main axes x, y, z of the virtual coordinate system with a common center point CP where the center marker 10 is located. The interface between the first end 71 of arm 70 and the ground plate 60 may allow a positioning along an axis, which is parallel to the respective (main) axis x, y, z of the coordinate system. With this respect, the orthogonal distance between a receptacle 65 of the ground plate 60 and the center point CP or center marker 10 corresponds to the orthogonal distance of a guiding portion 75 of the arm 70 and the respective satellite marker 11, 12, 13, 21, 22, 23, so that the satellite marker upon re-positioning changes the distance from the center marker 10, but does not leave the respective axis x, y, z. It should be noted that for the auxiliary markers or sub markers 21, 22, 23 the same applies, although their respective auxiliary axes are not provided with reference numerals.

FIG. 9 illustrates a reference body according to FIG. 8 applied to an anatomy 9 and illustrates different details, which can be established separately or in combination with respect to the reference body 1. For FIG. 9, the same applies as for FIG. 8.

FIG. 9 further illustrates that arm 70 can be adjusted with respect to ground plate 60 along a linear extension 65a of a receptacle 65 of the ground plate. For this purpose, the arm 70, in particular the first end 71 of arm 70 comprises a guiding portion 75, which has a linear extension 75a, which corresponds to the linear extension 65a of the receptacle 65. This linear extensions 65a and 75a are parallel to a respective axis x, y, z of the virtual coordinate system along which axis the center point CP and the center marker 10 are located as well as the respective satellite marker 11, 12, 13, 21, 22, 23. Thus, upon repositioning of the arm along the trajectory corresponding to the linear extensions 65a, 75a, only the distance of the respective satellite marker to the center marker 10 changes, but not the orientation of the respective axis x, y, Z.

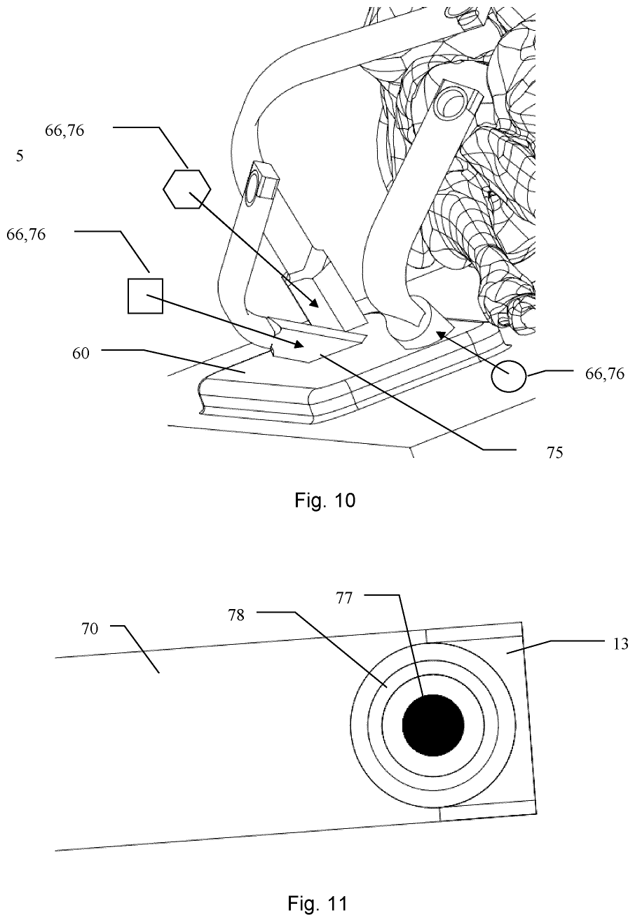

FIG. 9 further illustrates that a cross section 66 of a receptacle 65 of the ground plate 60 may be an unmistakable cross section, which allows only connection of a corresponding unmistakable counter cross section 76 of the arm 70, e.g., at its first end 71, in particular its guiding portion 75. Illustrative examples of unmistakable cross sections are illustrated in FIG. 9. Some of them may have a symmetry, if it can be expected that the surgeon generally applies the arm into the correct orientation. The illustrated circle may have a not illustrated rotational block allowing insertion only in a predetermined orientation. FIG. 10 illustrates the characteristic cross sections 66, 76 of the linear guiding portions 75 of the reference body arms 70 in detail. As can be seen in FIG. 10, the different cross sections allow only a particular type of arm to be inserted, so that unintentional changes can be avoided. It should be noted that the linear guiding portion 75 of the arm 70 and/or the linear receptacle 65 may be provided with a latching geometry allowing different defined intermediate positions. For this purpose, recesses and a ball detent may be provided. The purpose is to provisionally hold the arm while letting it slide in and out along an axis. There can be either a screw or ball detent (spring loaded ball bearing in a threaded canister) which automatically presses into the recess. Alternative embodiments may include a translational locking cam, thumb screw, over center latch, or another provisional retention means.

FIG. 9 further illustrates a radio dense marker arrangement on the arm 70, including a ring marker 78 and a line marker 77. The combination of a ring marker 78 and a line marker 77 allows monitoring of the viewing perspective. If the viewing position is in a way that ring marker 78 and line marker 77 are brought into an orientation that the front view of the line marker 77 is in the center of the ring marker 78, a predetermined viewing direction is achieved. In case the line marker 77 and the ring marker 78 are positioned as illustrated on the right arm in FIG. 9, the viewing direction is parallel to the respective axis of that arm. In case the line marker 77 and the ring marker 78 are positioned as illustrated on the left arm, the viewing direction is rather orthogonal to the respective axis of that arm. In case the viewing direction is aligned to the arm orientation, the line marker 77 and the ring marker 78 appear concentrical in a projection. This is illustrated in FIG. 11, where the line marker 77 is recognizable as a dot which is concentrical to the ling marker 78.

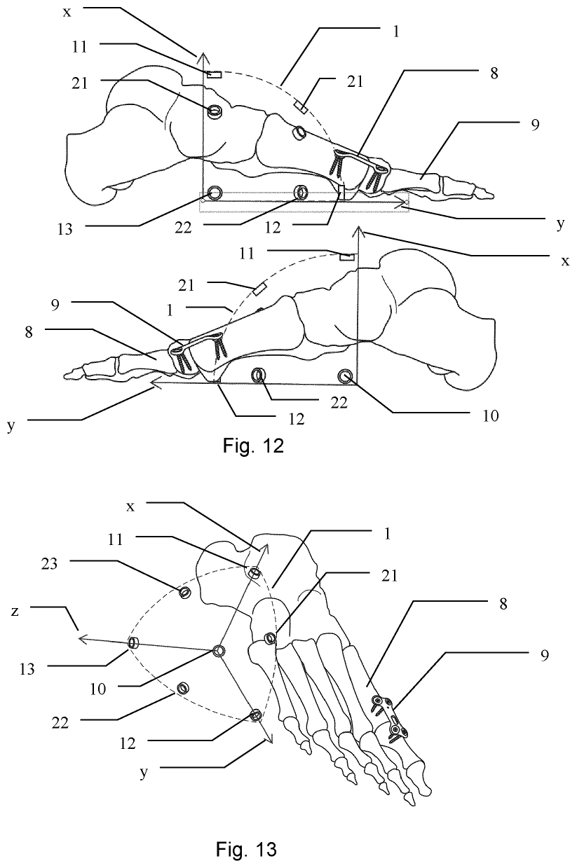

FIG. 12 illustrates the reference body 1 relative to an anatomy 9 and an implanted implant 8. As can be seen, the anatomy 9, here the anatomy of a foot with foot bones having implanted a bone plate implant 8 can be surrounded by the particular form of the reference body 1. This applies for both groups of embodiments, the group of embodiments relating to the partial spherical reference body as illustrated in FIGS. 1 to 7 and 12, as well as the embodiments relating to the arm design as illustrated in FIGS. 8 to 11. The reference body 1 positioned with respect to the anatomy 9 allows provision of reference markers. This includes radio dense markers 11, 12, 13, 21, 22, 23, as well as optical markers 31, 32, 33, although the latter ones are not illustrated in FIG. 12. In particular, the positioning of the reference body 1 with respect to the anatomy 9 allows provision of a reference coordinate system. Here a Cartesian coordinate system with axes x, y and z, which are represented by the main markers 11, 12, 13. If the relative position of the anatomy 9 and the reference body once is established, the spatial relative position of the reference body 1, the anatomy 9 and possible implants 8 can be derived from a single 2-dimensional image. Known relative position of radio dense markers 11, 12, 13, 21, 22, 23 as well as the optical markers 31, 32, 33 further allow a navigation by optical imaging (visible wavelength) instead of x-ray imaging once the spatial position of the reference body 1 and the anatomy 9 is established. This may reduce x-ray radiation exposure while maintaining the possibility to reliably navigate by optical imaging. Although not illustrated in detail this also applies for the embodiments of a reference body with an arm design as illustrated in FIGS. 8 to 11.

FIG. 13 illustrates schematically an x-ray view along the z-axis with the plane defined by the x-axis and the y-axis corresponding to the image plane. FIG. 13 illustrates overlapping images of an anatomy 9 with implanted implant 8 and the reference body 1 with radio dense or radiopaque markers 11, 12, 13 and features in a simulated monitor screen projection. As the relative position of the reference body 1 and the anatomy 9 is defined, a single image as illustrated in FIG. 13 may allow navigation during surgery only by optical imaging as described with respect to FIG. 12.

As described above, the reference body 1 may be utilized in many scenarios, such as prior to surgery as surgical planning tool, during surgery as a coordinating and live plan modification tool, and also after surgery as a diagnostic and measurement aid. For example, a first x-ray can be taken with reference body 1 positioned adjacent exemplary anatomy 9 prior to surgery as shown in FIG. 7. While reference body 1 is shown with ground plate 60 in FIG. 7, the first x-ray can be taken with only reference body 1 in other embodiments. A surgeon can then use the reference coordinate system provided by the reference body in the first x-ray to properly plan a surgical procedure such as implanting implant 8. For surgical procedures involving implanting bone plates such as implant 8, a trial or template can be placed at the target surgical site along with the reference body in a second x-ray. The second x-ray can then be used to precisely locate and secure implant 8. A third x-ray can then be taken with implanted implant 8 and the reference body as shown in FIG. 12 to verify proper implant placement. Thus, reference body 1 can be used in imaging prior to surgery (e.g., first x-ray), during surgery (e.g., second x-ray), and after surgery (e.g., third x-ray).

It should be noted, that the above-described details regarding the guiding trajectory according to the longitudinal extension 65a, 75a, the unmistakable cross section 66, 76 as well as the marker arrangement 77, 78 each contribute to the exactness of the reference device and that any combination thereof has beneficial and synergetic effects extending on the sum of the single effects.

### List of Reference Numbers

1 reference body
2 carrier structure of reference body
3 receptacle for marker
4 support structure
8 implant
9 exemplary anatomy
10 central marker/radio dense central marker
11 main satellite marker/radio dense main satellite marker
12 main satellite marker/radio dense main satellite marker
13 main satellite marker/radio dense main satellite marker
21 sub satellite marker/radio dense sub satellite marker
22 sub satellite marker/radio dense sub satellite marker
23 sub satellite marker/radio dense sub satellite marker
31 optical marker with unique pattern
32 optical marker with unique pattern
33 optical marker with unique pattern
60 reference body ground plate
65 receptacle for reference body arm in reference body ground plate
65a linear extension of receptacle
66 unmistakable cross-sectional shape of receptacle
70 reference body arm
71 one end/first end of reference body arm
72 other end/second end of reference body arm
75 linear guiding portion of reference body arm
75a linear extension of the linear guiding portion
76 unmistakable cross-sectional shape of reference body arm
77 line marker of (additional) radio marker on reference body arm
78 ring marker of (additional) radio marker on reference body arm
CP center point of virtual coordinate system/location of main marker on ground plate
d orthogonal distance from an axis/longitudinal extension of an item
deg degree
x main axis of virtual coordinate system/displacement axis for reference body arm
y main axis of virtual coordinate system/displacement axis for reference body arm
z main axis of virtual coordinate system/displacement axis for reference body arm

1. Reference body for providing a reproducible reference to predetermined reference axes of a virtual coordinate system for fluoroscopic/x-ray imaging, the reference body comprises:
a radio dense central marker located at a center of the virtual coordinate system,
a plurality of radio dense main satellite markers each located on one of the main axes of the virtual coordinate system, so that a line between the center marker and one of the main satellite markers represent a main axis of the virtual coordinate system.

2. Reference body according to claim 1, wherein the virtual coordinate system is a Cartesian coordinate system with orthogonal x, y and z axes, wherein each of the main satellite marker are provided on the positive branch of the main axes of x, y and z axes.

3. Reference body according to claim 1, further comprising a plurality of sub satellite markers, each provided on an equally divided angles between two positive branches out of the x, y and z axes, wherein in particular the equally divided angle is 45 deg or 30 deg.

4. Reference body according to any claim 1, wherein at least a part, in particular all satellite markers are equally distant from the center marker.

5. Reference body according to claim 1, wherein the center marker has a radio dense shape of a spatial cross with cross lines along the main axes of the virtual coordinate system.

6. Reference body according to claim 1, wherein at least one of the satellite markers has a radio dense shape of a ring with a ring opening aligned to center marker and/or a radio dense shape of cross of an extension traverse to the direction toward the center marker.

7. Reference body according to claim 1, wherein at least a part of the satellite marker has locally allocated optical markers, wherein in particular each of the optical markers has a unique optical pattern allowing identification as well as determination of a position of the respective satellite marker.

8. Reference body according to claim 1, further comprising a reference body ground plate and a plurality of reference body arms,
wherein the center marker is fixed to the ground plate at a predefined center position corresponding to a center point of the virtual coordinate system,
wherein each of the plurality of arms has a linear guiding portion at the one end of one of the plurality of arms,
wherein one of the satellite markers is provided in a predefined orthogonal distance from the linear extension of the linear guiding portion at the other end of the respective one of the plurality of arms,
wherein the ground plate comprises a plurality of receptacles with a linear extension for receiving a respective linear guiding portion of one of the arms,
wherein each of the linear extensions of the receptacles is offset by a predefined orthogonal distance from the center position of the center marker, so that a satellite marker positioned at the respective predefined orthogonal distance from the linear extension of the linear guiding portion is displaceable along a linear trajectory running through the center position of the center marker.

9. Reference body according to claim 8, wherein each of the receptacles has an unmistakable cross sectional shape representative for an orthogonal distance of the center marker from the linear extension of said receptacle for unmistakable receiving a linear guiding portion of one of the reference body arms with a corresponding cross sectional shape representative for an orthogonal distance of the satellite marker on said arm from the linear extension of the linear guiding portion.

10. Reference body according to claim 8, wherein the receptacles are through bores along axes for distance adjustment of the respective satellite marker on the respective reference body arm.

11. Reference body according to claim 8, wherein the receptacle and the reference body arm have a corresponding color code representative for the orthogonal distance of the center portion from a linear extension of the receptacle and the satellite marker from the linear extension of the linear guiding portion of the reference body arm.

12. Reference body according to claim 8, wherein the reference body arm comprises an additional radio marker arrangement with a line marker along the axis of a linear extension of the linear guiding portion and a ring marker concentric to the axis of the linear extension of a linear guiding portion and distant from the line marker along the axis of the linear extension of a linear guiding portion.

13. Reference body ground plate, the ground plate comprises:
a radio dense center marker located at center position representing a center of a virtual coordinate system,
a plurality of receptacles with a linear extension for receiving a linear guiding portion of an reference body arm to be connected to the ground plate,
wherein each of the linear extensions of the receptacles is offset parallel in a predefined orthogonal distance from one of the main axes of the virtual coordinate system, so that a satellite marker positioned at a respective predefined orthogonal distance from the extension of the linear guiding portion of a reference body arm to be connected to the ground plate is displaceable along the respective main axis of the virtual coordinate system.

14. Reference body according to claim 13, wherein each of the receptacles has an unmistakable cross sectional shape representative for an orthogonal distance of the center marker from the linear extension of said receptacle for unmistakable receiving a linear guiding portion of a reference body arm with a corresponding cross sectional shape representative for an orthogonal distance of a satellite marker on said reference body arm from a linear extension of the linear guiding portion of said reference body arm to be connected to the reference body ground plate.

15. Reference body according to claim 13, wherein the receptacles are through bores along axes for distance adjustment of a satellite marker on a reference body arm to be connected to the reference body ground plate.

16. Reference body according to claim 13, wherein the receptacle and a reference body arm to be connected have a corresponding color code representative for the orthogonal distance of the center portion from a linear extension of the receptacle and a satellite marker from a linear extension of a linear guiding portion of a reference body arm to be connected.

17. Reference body arm for positioning a satellite marker with respect to a ground plate to which the reference body arm is to be connected, the reference body arm comprises:
a linear guiding portion to be received in a receptacle of a ground plate,
a radio dense satellite marker positioned at a predefined orthogonal distance from a linear extension of the linear guiding portion of the reference body arm.

18. Reference body according to claim 17, wherein the linear guiding portion has an unmistakable cross sectional shape representative for the orthogonal distance of the satellite marker from the linear extension of the linear guiding portion for unmistakable to be received by a linearly extending receptacle of a reference body ground plate with a corresponding cross sectional shape representative for a corresponding orthogonal distance of a linear extension of the linearly extending receptacle from a center portion of the reference body ground plate to which the reference body arm is to be connected.

19. Reference body according to claim 17, wherein the reference body arm and a receptacle to which the reference body arm is to be connected have a corresponding color code representative for the orthogonal distance of the satellite marker from the linear extension of the linear guiding portion of the reference body arm and a center portion of a reference body ground plate to which the reference body arm is to be connected from a linear extension of a receptacle of a reference body ground plate to which the reference body arm is to be connected.

20. Reference body according to claim 17, further comprising an additional radio marker arrangement with a line marker along the axis of a linear extension of the linear guiding portion and a ring marker concentric to and distant along the axis of the linear extension of a linear guiding portion from the line marker.

21. A method for providing for an anatomy and a bone implant a reproducible reference to predetermined reference axes of a virtual coordinate system for fluoroscopic/x-ray imaging, the method comprises:
positioning with respect to each other the anatomy, the bone implant and a reference body having a radio dense central marker located at a center of the virtual coordinate system, and a plurality of radio dense main satellite markers each located on one of the main axes of the virtual coordinate system, so that a line between the center marker and one of the main satellite markers represent a main axis of the virtual coordinate system,
x-ray imaging the anatomy, the bone implant and the reference body from at least two different viewing angles allowing for precise reference and interpolation of a 2D image taken along a 3D axis.

22. The method of claim 21, wherein x-ray imaging comprises x-ray imaging from at least three different viewing angles.

23. The method of claim 21, wherein x-ray imaging comprises x-ray imaging from at least three different viewing angles in a first plane.

24. The method of claim 21, wherein x-ray imaging comprises x-ray imaging from at least two different viewing angles in a first plane and at least two different viewing angles in a second plane.

25. The method of claim 24, wherein the first plane and the second plane are orthogonal with respect to each other.

26. The method of claim 21, wherein the virtual coordinate system is a Cartesian coordinate system with orthogonal x, y and z axes, wherein each of the main satellite marker are provided on the positive branch of the main axes of x, y and z axes.

27. The method of claim 21, wherein x-ray imaging is carried out upon targeting at least one of the satellite markers aligned to the center marker.

28. The method of claim 21, further comprising identifying at least one optical marker each having a unique optical pattern and being allocated to a radio dense satellite marker, determining a position of the respective satellite marker based on the respective identified optical marker, and determining a position of a bone implant relative to the at least one optical marker based on the determined position of the respective satellite marker.

29. The method of claim 21, further comprising identifying at least one optical marker each having a unique optical pattern and being allocated to a radio dense satellite marker, determining a position of the respective satellite marker based on the respective identified optical marker, and determining a position of a bone plate and at least one bone screw of the bone implant relative to the at least one optical marker based on the determined position of the respective satellite marker.

30. The method of claim 21, further comprising positioning of the anatomy on a reference body ground plate having implemented the center marker at a predefined center position corresponding to a center point of the virtual coordinate system and having a plurality of receptacles with a linear extension,
positioning of a plurality of reference body arms each having a linear guiding portion with a linear extension at one end of the respective reference body arm for being received in a respective receptacle and a respective one of the satellite markers provided in a predefined orthogonal distance from the linear extension of the linear guiding portion at the other end of the respective one of the plurality of arms, each of the linear extensions of the receptacles is offset by a predefined orthogonal distance from the center position of the center marker, so that a satellite marker positioned at the respective predefined orthogonal distance from the linear extension of the linear guiding portion is displaceable along a linear trajectory running through the center position of the center marker.

31. The method of claim 21, wherein x-ray imaging is carried out upon targeting a line marker provided along an axis of a linear extension of the linear guiding portion and a ring marker concentric to the axis of the linear extension of a linear guiding portion and distant from the line marker along the axis of the linear extension of a linear guiding portion, so as to achieve an x-ray image where the ring marker and the line marker are concentric in the x-ray image.

32. A method for imaging an anatomy with reference to a virtual coordinate system, the method comprising the steps of:
positioning a reference body adjacent an anatomy, the reference body having a radio dense central marker and at least three radio dense main satellite markers disposed about the central marker, the central marker defining a center of virtual coordinate system;
imaging the anatomy and the reference body from a first viewing angle to generate a first 2D image;
imaging the anatomy and the reference body from a second viewing angle to generate a second 2D image, the second viewing angle being different from the first viewing angle, and
interpolating any of the first and second 2D images along the virtual coordinate system, wherein lines joining each of the three main satellite markers with the central marker form main axes of the virtual coordinate system.

33. The method of claim 32, wherein the step of positioning the reference body adjacent the anatomy includes positioning an anatomy with an implant.

34. A method for implant placement, the method comprising the steps of:
imaging an anatomy and a reference body placed adjacent the anatomy to generate a first image with a virtual coordinate system, the reference body having a radio dense central marker and at least three radio dense main satellite markers disposed about the central marker, the central marker defining a center of virtual coordinate system, and
placing an implant at a target location on the anatomy based on the first image,
wherein lines joining each of the three main satellite markers with the central marker form main axes of the virtual coordinate system.

35. The method of claim 34, further including a step of imaging the anatomy with the implant and the reference body placed adjacent the anatomy to generate a second image with the virtual coordinate system.

36. A method for implant placement, the method comprising the steps of:
imaging an anatomy and a reference body placed adjacent the anatomy to generate a first image with a virtual coordinate system, the reference body having a radio dense central marker and at least three radio dense main satellite markers disposed about the central marker, the central marker defining a center of virtual coordinate system;
placing an implant trial at a target location on the anatomy based on the first image;
imaging the anatomy with the implant trial and the reference body placed adjacent the anatomy to generate a second image with the virtual coordinate system, and
placing an implant at the target location on the anatomy based on the second image,
wherein lines joining each of the three main satellite markers with the central marker form main axes of the virtual coordinate system.
